(19) United States
(12) Patent Application Publication
(10) Pub. No.: US 2016/0271120 A1
(43) Pub. Date: Sep. 22, 2016

(54) PHARMACEUTICAL COMPOSITION FOR CLEANSING OF THE GASTROINTESTINAL TRACT

(71) Applicant: Oakdene Holdings LLC, Mendham, NJ (US)

(72) Inventors: Aman Trehan, Somerset, NJ (US); James Garegnani, Somerset, NJ (US)

(73) Assignee: Oakdene Holdings LLC, Mendham, NJ (US)

(21) Appl. No.: 15/052,502

(22) Filed: Feb. 24, 2016

Related U.S. Application Data
(60) Provisional application No. 62/134,214, filed on Mar. 17, 2015.

(51) Int. Cl. A61K 31/4402; A61K 47/02; A61K 9/00; A61K 31/765

(52) U.S. Cl. CPC A61K 31/4402; A61K 31/765; A61K 47/02; A61K 9/0095

(57) ABSTRACT

A pharmaceutical composition for cleansing the gastrointestinal tract prior to a colonoscopy or other gastrointestinal procedure is provided. The composition comprises polyethylene glycol and sodium picosulfate and is suitable to make up to 1.5 liters of aqueous solution for oral administration.

### CROSS-REFERENCE TO RELATED APPLICATIONS

This application is a nonprovisional application claiming the benefit priority of U.S. Provisional Application No. 62/134,214, filed Mar. 17, 2015, the contents of which are incorporated herein in their entirety by reference.

### BACKGROUND OF THE INVENTION

(a) Field of the Invention

The present invention is directed to a pharmaceutical composition for cleansing the gastrointestinal tract prior to a colonoscopy or other gastrointestinal procedure. In particular, the present invention provides a bowel cleansing or colon lavage composition comprising polyethylene glycol and sodium picosulfate.

(b) Description of the Related Art

Cleansing of the gastrointestinal tract (including the bowel and colon) is important prior to numerous diagnostic and surgical procedures, for example before colonoscopy, barium enema examination or colon surgery. It is also useful for preventing infection after surgery on the lower intestine. Colon cleansing is also known as colon clearing.

A variety of methods for colon cleansing are known. For example, dietary manipulation, laxatives, cathartics and enemas have been traditionally used (Thomas, G. et al., Gastroenterology, 1982, 82, 435 437). Sodium phosphate solutions (Clarkston, W. K. et al., Gastrointestinal Endoscopy, 1996, 43, 43 48) and magnesium citrate/sodium picosulphate solutions (Regev, A. et al., Am. J. Gastroenterol., 1998, 93, 1478 1482) have also been used.

Those methods suffer from various drawbacks. Dietary manipulation and laxatives are time consuming; enemas are unpleasant for the patient; and dangerous salt and water losses may occur with cathartics, enemas and sodium phosphate solutions.

Sodium phosphate solutions, such as that available from the C.B. Fleet Company Inc. (4615 Murray Place, PO Box 11349, Lynchburg, Va. 24506, USA) under the trade name Phospho-soda™ are hyperosmotic solutions which increase retention of water in the intestine and thereby promote bowel movement. Phospho-soda™ comprises, per 5 ml portion, 2.4 gm monobasic sodium phosphate monohydrate with 0.9 gm dibasic sodium phosphate heptahydrate in a buffered aqueous solution. Typically, 20 to 45 ml are taken by an adult patient followed by a large quantity of water. If the water is not taken, elevated serum sodium and phosphate levels may result, leading to serious kidney problems. The risk of those side effects makes it necessary for there to be direct medical supervision during administration of Phospho-soda™.

Another approach to colon cleansing is orthostatic intestinal lavage, in which a large volume of an electrolyte solution is ingested, either by drinking or by infusion through a nasogastric tube. Such lavage solutions are also known as bowel lavage solutions. Consumption of the solution results in volume-induced diarrhoea and thus cleansing of the colon. The method is generally faster than the traditional approaches. The main component of early lavage solutions was sodium chloride. However, as a significant percentage of such saline-based lavage solutions is absorbed into the bloodstream in the gut of the patient, a rapid increase in intravascular volume results, which has caused serious complications in some patients.

In 1980, Davis and co-workers reported the development of a lavage solution that they described as being associated with minimal water and electrolyte absorption or secretion (Davis G. R. et al., Gastroenterology, 1980, 78, 991 995). The solution included sodium sulphate and polyethylene glycol. Sulphate ions are poorly absorbed in the gut. As a result, sodium absorption is markedly reduced when sulphate, rather than chloride or bicarbonate, is the predominant counter-anion present in a lavage solution in the gut. In addition to sodium sulphate (40.0 mM, 5.68 gm/l), the solution described by Davis et al. comprises sodium chloride (25 mM, 1.463 gm/l), potassium chloride (10 mM, 0.745 gm/l), sodium bicarbonate (20 mM, 1.680 gm/l), polyethylene glycol (PEG 4000 “carbowax”, 64 gm/l) and water. The solution was administered in a quantity of 4 liters. The solution was shown to be effective in cleansing the gastrointestinal tract and it has been commercialised under the trade name GoLYTELY™ (Braintree Laboratories Inc., Braintree, Mass., U.S.A.). The commercially available GoLYTELY™ composition, also known as Klean Prep™, has been available since August 1996 and at the time of filing, is supplied in dry powder form comprising sodium sulphate (40.0 mM, 5.685 gm/l), sodium chloride (25 mM, 1.464 gm/l), potassium chloride (10 mM, 0.743 gm/l), sodium bicarbonate (20 mM, 1.685 gm/l) and PEG 3350 polyethylene glycol (59 gm/l) for making up to 4 liters. GoLYTELY™ is also supplied as an aqueous solution.

The GoLYTELY™ solution, whilst effective, has a very salty taste, which adversely affects patient compliance. Typically the composition is presented as 4 or more liters of aqueous solution, and it is important that the whole prescribed volume be consumed.

Fordtran et al. (PCT Pub. No. WO 87/00754) subsequently developed a reduced sodium sulphate solution (RSS) comprising no sodium sulphate but instead having a relatively high concentration of polyethylene glycol (75 to 300 gm/l). The solution is also administered in a quantity of 4 liters and commercialised by Braintree Laboratories Inc. (Braintree, Mass., U.S.A.) under the brand name NuLYTELYT™. The NuLYTELY™ composition contains PEG 3350 (105 gm/l), sodium bicarbonate (1.43 gm/l), potassium chloride (0.37 gm/l) and sodium chloride (2.80 gm/l) and it is supplied in a dry powder form for making up to 4 liters.

Although being effective in colon cleansing in the clinic, both the GoLYTELY™ and NuLYTELY™ solutions must be ingested in large quantities. In spite of the absence of sodium sulphate in NuLYTELY™, both NuLYTELY™ and GoLYTELY™ have an unpleasant salty taste. The unpleasant taste exacerbates the problem of patient compliance, particularly when the patient is not under medical supervision.

PCT Pub. No. WO 89/05659 (Borody et al.) discloses an orthostatic lavage solution comprising polyethylene glycol, electrolytes and from 0.25 to 50 gm/l ascorbic acid (vitamin C) or a salt thereof. The presence of ascorbic acid or a salt thereof is said to reduce the required volume of the solution to 3 liters or less. Whilst about 3 gm of ascorbic acid may be absorbed in the intestine (Hornig, D. et al., Int. J. Vit. Nutr. Res., 1980, 50, 309) any further ascorbic acid is reported in WO 89/05659 to contribute to the diarrhoea and to inhibit bacterial gas generation and bacterial reproduction. The ascorbic acid is also said to facilitate ingestion of the lavage solution because its pleasant acidic taste masks the usual nauseating taste of the salty polyethylene glycol solution.

A formulation as described by Borody has been available on the market in Australia for more than 10 years under the trade name GLYCOPREP™ C (Pharmatel). The GLYCOPREP™ C dry composition comprises PEG 3350 (53 gm/l), sodium chloride (2.63 gm/l), potassium chloride (0.743 gm/l), sodium sulphate (5.6 gm/l), ascorbic acid (6 gm/l), aspartame (0.360 gm/l), citric acid (0.900 gm/l) and lemon flavour (0.090 gm/l). Three liters of the solution are generally administered. Although the addition of ascorbic acid may provide an improved bowel preparation, the preparation nonetheless needs to be ingested in quantities of about 3 liters.

Typical gastrointestinal lavage formulations are described in U.S. Pat. App. No. 2007/0098764, U.S. Pat. App. No. 2007/0298008, Fordtran et al. Gastroenterology 1990: 98:11-16, Ernstoff et al. Gastroenterology 1983: 84:1512-6, Davis et al. Gastroenterology. 1980: 78:991-995, herein incorporated by reference in their entireties.

U.S. Pat. Nos. 7,169,381 and 7,658,914 disclose a cleansing solution comprising an alkali metal or alkaline earth metal sulphate, ascorbic acid and/or one or more salts thereof, a relatively high concentration of PEG and, optionally, further electrolytes. The solution has a cleansing action that is effective when administered in a small volume, and is palatable. The product based on these patents is commercialised by Salix Pharmaceuticals, Inc. under the brand name MoviPrep™.

U.S. Pat. No. 8,778,306 discloses a kit comprising a plurality of individually packaged doses of a palatable osmotic agent that are to be administered in an aqueous solution and with a plurality of doses of electrolytes such as sodium bicarbonate, sodium chloride, sodium sulfate, potassium chloride, ascorbic acid and/or sodium ascorbic acid, magnesium salts. The preferred osmotic agent is polyethylene glycol. According to the patent, the plurality of doses of electrolytes comprises pills, capsules, tablets, gel-caps, gel-caps filled with a paste or suspension, and micro-encapsulated salts for administration as a capsule or suspended in a liquid.

The compositions of the prior art containing polyethylene glycol, sodium sulfate, electrolytes, salts and acids are summarised in Tables 1-3. The calculated osmolarity of the solutions (in mOsmol/kg) is also given in the table together with the recommended dose (in liters).

TABLE 1
PEG and Sodium Sulfate-Containing Purgatives

| | Total Qty in gms per Liter | | | | | | | |
|---|---|---|---|---|---|---|---|---|
| Product | PEG | Na₂SO₄ | NaHCO₃ | CNaCl | KCl | CVit C | Osm | Volume |
| Kleanprep | 59 | 5.68 | 1.68 | 1.465 | 0.74 | — | N/A | 4 |
| GoLYTELY | 15 | 1.4 | 0.5 | 0.4 | 0.19 | — | 255 | 4 |
| NuLYTELY | 26 | — | 0.4 | 0.7 | 0.9 | — | 176 | 4 |
| Glycoprep C | 18 | 1.4 | — | 0.66 | 0.19 | 1.5 | 291 | 3 |
| MoviPrep | 100 | 7.5 | optional | 2.7 | 1 | 10.6 | 300-700 | 2 |

TABLE 2
Sodium Picosulfate-Containing Purgatives

| | Total Qty in gms | | | | | | |
|---|---|---|---|---|---|---|---|
| | | | Citric | | Saccharin | Orange | |
| Product | SPS | MgO | acid | KHCO₃ | Na | Flavor | Volume |
| Picolax | 20 | 7 | 24 | 1 | 0.12 | 0.12 | 2 |
| (2 | | | | | | | |
| Sachets) | | | | | | | |
| Prepopik | 20 | 7 | 24 | 1 | 0.12 | 0.12 | 0.8 |
| (2 | | | | | | | |
| Sachets) | | | | | | | |

TABLE 3
Sodium Picosulfate and PEG-Containing Purgative
[Available in New Zealand from Fresenius Kabi]

| | Total Qty in gms | | | | | | | | | | |
|---|---|---|---|---|---|---|---|---|---|---|---|
| | Glycoprep C* - 1 Sachet | | | | | | | PicoPrep - 2 Sachets | | | |
| | | Citric | Na | | | | | | Citric | | |
| Product | PEG | acid | Sulfate | NaCl | KCl | Vit C | Volume | SPS | acid | MgO | Volume |
| Prepkit C | 23.4 | 0.4 | 2.5 | 1.15 | 0.3 | * | 1 | 89.6 | 108 | 31.5 | 0.5 |
| (Glycoprep C + | | | | | | | | | | | |
| PicoPrep) | | | | | | | | | | | |

*Quantity not available.

Overall, the currently available products are believed to suffer from one or more issues such as: (a) requirement to be ingested in large volumes; (b) ineffective if administered in lower volumes than recommended; (c) require complex regimen of administration; and (d) unpleasant salty taste. Particularly, ingestion of large volumes of gut lavage solution as prescribed for several prior art compositions is still generally physically unpleasant or, for some patients, even impossible, may result in retching, and is time consuming.

There exists a need for bowel cleansing or lavage compositions which can be used to prepare low volume solutions, effective in bowel cleansing, that also possess a pleasant taste.

### SUMMARY OF THE INVENTION

The present invention provides a pharmaceutical composition for cleansing the gastrointestinal tract. The composition comprises an admixture of polyethylene glycol, sodium picosulfate and one or more electrolytes. In one aspect, the composition can consist essentially of an admixture of polyethylene glycol, sodium picosulfate and one or more electrolytes. In another aspect, the composition can consist of an admixture of polyethylene glycol, sodium picosulfate and one or more electrolytes.

In one aspect, there is provided a pharmaceutical composition for cleansing the gastrointestinal tract, per up to 1.5 liter of aqueous solution to be made, comprising an admixture of: (a) polyethylene glycol; (b) sodium picosulfate; and (c) one or more electrolytes. In one embodiment of this aspect, the composition can consist essentially of an admixture of polyethylene glycol, sodium picosulfate and one or more electrolytes. In another aspect, the composition can consist of an admixture of polyethylene glycol, sodium picosulfate and one or more electrolytes.

The composition may further comprise at least one inorganic acid and/or organic acid, or mixtures thereof.

In another aspect, these is provided a pharmaceutical composition for cleansing the gastrointestinal tract in a single packed dose, per up to 1.5 liter of aqueous solution to be made, comprising an admixture of: (a) polyethylene glycol; (b) sodium picosulfate; and (c) one or more electrolytes.

In another aspect, the dose of the pharmaceutical composition of the invention is provided in the form of powder, granules, pills, sachets, capsules, tablets, gel-caps, gel-caps filled with a paste or suspension, micro-encapsulated salts for administration as a capsule or suspended in a liquid. Preferably, the dose of the bowel cleansing composition is provided in the form of a sachet.

The electrolytes in the composition may be selected from sodium chloride, potassium chloride, sodium hydrogen carbonate, sodium sulfate, sodium carbonate, salts of magnesium, such as magnesium sulfate or magnesium citrate.

In another aspect, the pharmaceutical composition of the invention is reconstituted, mixed and/or dissolved in an aqueous solution and made up to 1.5 liters, preferably up to 1 liter before administration, and then administered.

In another aspect, there is provided an aqueous solution of up to a 1.5 liter volume for cleansing the gastrointestinal tract comprising: (a) polyethylene glycol; (b) sodium picosulfate; and (c) one or more electrolytes.

In another aspect, reconstitution of the admixture of the pharmaceutical composition of the invention in an aqueous solution provides a solution that exhibits a pH in the range of from about 3.0 to about 6.0, and preferably in the range of about 3.0 to about 4.0.

In another aspect, the pharmaceutical composition of the invention is at least about 30%, at least about 50%, or at least about 70% more palatable than the solution in which pH is not adjusted to be in the range of from about 3.0 to about 4.0.

In another aspect, the bowel cleansing aqueous solution of the invention has a total volume of less than 1.5 liter. In one embodiment the bowel cleansing aqueous solution of the invention has a total volume of about 1 liter or less.

In another aspect, the amount of polyethylene glycol in the pharmaceutical composition or aqueous solution of the invention is present in the range of about 25 gm to about 350 gm.

In another aspect, the pharmaceutical composition of the invention or the aqueous solution of the invention contains sodium picosulfate being present in the range of about 40 gm to about 200 gm.

In another aspect, the pharmaceutical composition or aqueous solution of the invention is administered in a single dose, preferably in a series of doses, for example, two, three or four doses.

The inorganic acid and/or organic acid in the pharmaceutical composition or aqueous solution may be selected from the group consisting of citric acid, acetic acid, ascorbic acid, phosphoric acid, malic acid, succinic acid, formic acid, fumaric acid, maleic acid, adipic acid, butyric acid, glyconic acid, lactic acid, oxalic acid, tartaric acid and mixtures thereof.

In another aspect, the pharmaceutical composition or aqueous solution of the invention is devoid of ascorbic acid, one or more salts of ascorbic acid, or mixtures thereof.

In another aspect, the pharmaceutical composition or aqueous solution of the invention further comprises an alkali metal or alkaline earth metal sulphate or a mixture of an alkali metal or alkaline earth metal sulphates.

In another aspect, the reconstituted solution containing the pharmaceutical composition of the invention has an osmolarity within the range of from 300 to 700 mOsmol/kg.

In another aspect, there is provided a method of cleansing the gastrointestinal tract prior to an endoscopy including colonoscopy and sigmoidoscopy, a barium enema examination, capsule endoscopy, colon surgery or gastrointestinal tract surgery, wherein the method comprises orally administering the pharmaceutical composition or aqueous solution as substantially described herein.

The gastrointestinal tract may be cleansed prior to carrying out a diagnostic, therapeutic and/or surgical procedure on the patient. For example, the gastrointestinal tract is cleansed prior to an endoscopy, such as a colonoscopy or sigmoidoscopy. The gastrointestinal tract may be cleansed prior to a barium enema examination, capsule endoscopy, colon surgery or gastrointestinal tract surgery.

Still other aspects and advantages of the invention will be apparent from the following detailed description of the invention.

### DETAILED DESCRIPTION OF THE INVENTION

The present invention provides a composition for cleansing the gastrointestinal tract of a patient using the composition prior to a diagnostic, surgical or therapeutic procedure. The composition of invention requires a low volume of liquid, particularly an aqueous solvent to prepare a solution for cleansing the gastrointestinal tract (including the bowel and/or colon), which low volume is believed to improve patient compliance. The composition has acceptable palatability that can be attributed to improved taste and thus may further enhance patient compliance. An additional advantage of the composition is its simple administration regimen.

The present invention provides a pharmaceutical composition for cleansing the gastrointestinal tract comprising polyethylene glycol and sodium picosulfate. It was surprisingly found that the volume of the composition can be made into a maximum of up to 1.5 liters of an aqueous solution upon reconstituting in a liquid, preferably an aqueous solution or water, while simultaneously maintaining the cleansing effect of the composition that is comparable to the effect achieved by administering larger volumes of the prior art compositions. The reduction in the total volume of liquid to be consumed for gastrointestinal tract cleansing has been found to significantly improve patient compliance.

It was surprisingly found that a cleansing solution comprising polyethylene glycol, sodium picosulfate, one or more electrolytes and optionally, at least one inorganic acid and/or organic acid, or mixtures thereof in legitimate concentration, has a cleansing action that is effective when administered in a small volume, and the resulting composition is also palatable. The cleansing solution comprising a composition of the invention achieves satisfactory colon cleansing when used in a quantity of less than 1.5 liters. In comparison, conventional cleansing solutions must be used in a quantity of up to 4 liters.

The invention provides a composition for admixture with water wherein the composition comprises, per less than 1.5 liter of aqueous solution to be made, the following components: polyethylene glycol, sodium picosulphate, one or more electrolytes, and optionally at least one inorganic acid and/or organic acid, or mixtures thereof. Preferably, the pharmaceutical composition is provided in a single packed dose.

Preferably, the pharmaceutical composition of the invention is reconstituted, mixed and/or dissolved in an aqueous solution and made up to about 1.5 liters, preferably up to about 1 liter before administration.

In an embodiment, a bowel cleansing aqueous solution of up to a 1.5 liter volume, comprises: (a) polyethylene glycol; (b) sodium picosulfate; (c) one or more electrolytes; and (d) optionally, at least one inorganic acid and/or organic acid, or mixtures thereof.

The volume of the bowel cleansing aqueous solution of the invention is less than 1.5 liter and preferably the volume is about 1 liter or less.

The solutions of the invention may be isotonic or hyperosmotic. Preferably, the solution is not iso-osmotic, i.e., it does not have the same osmotic pressure as the blood in the gut vasculature. The solutions are, however, approximately iso-osmolar, that is to say, the solution excreted from the patient has substantially the same ion content as the solution ingested. Consequently, there is no substantial net change in the ion levels in the blood of the patient. As one of skill in the art would readily understand, this provides advantages to individuals being administered solutions of the invention.

In an embodiment, the components of the composition are selected such that an aqueous solution made from up to 1.5 liters has an osmolarity within the range of from 300 to 700 mOsmol/kg. The osmolarity of a solution can be measured using standard laboratory techniques. It can also be calculated from a knowledge of the components of a solution.

In a further embodiment, reconstitution of the admixture of the pharmaceutical composition in aqueous solution provides a solution that exhibits a pH in the range of from about 3.0 to about 6.0, and preferably in the range of about 3.0 to about 4.0. It was also surprisingly found that the taste (particularly the salty taste) of the solution may improved significantly by adjusting the pH of the solution within the pH range of about 3.0 to about 4.0.

In a further embodiment, the pharmaceutical composition is at least about 30%, at least about 50%, or at least about 70% more palatable than the solution in which pH is not adjusted in the range of from about 3.0 to about 4.0.

When adjusting the pH of the composition, the pH may be increased or decreased by at least about 0.05 pH units, at least about 0.1 pH units, at least about 0.15 pH units, at least about 0.2 pH units, at least about 0.3 pH units, at least about 0.4 pH units, or at least about 0.5 pH units.

The desired pH range is from about 3.0 to about 6.0, preferably from about 3.0 to about 4.0, from about 3.1 to about 3.9, from about 3.2 to about 3.8, from about 3.3 to about 3.7, from about 3.4 to about 3.6, or about 3.5.

The composition further may contain one or more compounds for pH adjustment including, but not limited to, ammonium hydroxide, sodium carbonate, potassium carbonate, sodium bicarbonate, carbon dioxide, and mixtures thereof.

The polyethylene glycol (PEG) used in a composition of the present invention preferably has an average molecular weight of 2000 or greater. Preferably the PEG has an average molecular weight of 2500 or greater. Preferably the PEG has an average molecular weight of 4500 or lower. For example the PEG may be PEG 3350 or PEG 4000. Optionally, the PEG used in compositions of the invention may comprise two or more different PEG species.

The amount of polyethylene glycol in the pharmaceutical composition or aqueous solution of the invention is present in the range of about 25 gm to about 350 gm. The composition of the invention preferably comprises 25 gm or more of PEG per liter, more preferably 30 gm or more of PEG per liter. Preferably, compositions of the invention comprise 80 gm or less of PEG per liter, more preferably 150 gm or less of PEG per liter, still more preferably 100 gm or less of PEG per liter, still more preferably 125 gm or less of PEG per liter. For example, a composition of the present invention may comprise PEG at a concentration within a range wherein the lower limit is 25 or 100 gm per liter and the upper limit is, independently, 350, 250, 150 or 125 gm per liter.

The amount of sodium picosulfate in the pharmaceutical composition or aqueous solution of the invention is present in the range of about 40 gm to about 200 gm.

Suitable electrolytes for use in the composition of the invention comprise one or more metal salts, anions (e.g., bicarbonate, chloride, phosphate, sulfate, etc.), or cations (e.g., sodium, potassium, magnesium, calcium, etc.). Examples of electrolytes include, but are not limited to, sodium bicarbonate, sodium chloride, potassium chloride, sodium sulfate, sodium bicarbonate, sodium chloride, sodium phosphate (e.g. monosodium phosphate, disodium phosphate, trisodium phosphate, etc.), potassium bicarbonate, potassium phosphate, potassium sulfate, magnesium sulfate, magnesium oxide, magnesium bicarbonate, magnesium chloride, magnesium phosphates, calcium bicarbonate, calcium chloride, calcium phosphate, and calcium sulfate, etc.

Suitable inorganic acids or an organic acids for use in the composition of the invention include, but are not limited to, citric acid, acetic acid, ascorbic acid or salts thereof, phosphoric acid, malic acid, succinic acid, formic acid, fumaric acid, maleic acid, adipic acid, butyric acid, glyconic acid, lactic acid, oxalic acid, tartaric acid and mixtures thereof, or other permitted food acids.

In one embodiment, the composition is devoid of ascorbic acid, or its salt or mixture thereof. In contrast to the prior art teaching it was surprisingly found that without using ascorbic acid or a salt thereof in the composition, it is still possible to reduce the required volume of solution to less than 1.5 liters.

In an embodiment, the composition may be a liquid (e.g., aqueous solution) or a solid (e.g., powder or granule mixture).

A composition in accordance with the invention may be in the form of a powder, granules, pills, sachets, capsules, tablets, gel-caps, gel-caps filled with a paste or suspension, micro-encapsulated salts for administration as a capsule or suspended in a liquid or any other suitable physical form. A composition of the invention is preferably provided in unit dosage form, for example, in a sachet.

The pharmaceutical composition of the present invention is administered orally, and may be used to prepare (e.g., cleanse or clear) any part(s) of the gastrointestinal tract, including, but not limited to, the oesophagus, stomach, intestine (or bowel) such as the small intestine and the large intestine including cecum, colon and rectum. For example, the present methods may be used to empty the bowel.

In an embodiment, the pharmaceutical composition or aqueous solution of the invention is administered in a single dose.

In a further embodiment, the pharmaceutical composition or aqueous solution of the invention is administered in a series of doses, for example, two, three or four doses.

The composition may be administered over a time period ranging from about 30 minutes to about 3 days, from about 1 hour to about 24 hours, from about 2 hours to about 12 hours, or from about 1 hour to about 4 hours. The administration time period may be in a continuous period or a discontinuous period. In discontinuous administrations, a portion of the composition, for example, approximately half, may be administered the evening before the diagnostic, therapeutic or surgical procedure is to be carried out, with the remainder of the composition being administered on the day of the procedure. The composition may be taken once or several times per day on the day of the diagnostic, surgical or therapeutic procedure, and/or on the day(s) preceding the procedure, depending upon various factors, such as the procedure, the degree of cleansing required, the patient's condition (e.g., the presence of complicating bowel conditions such as constipation).

It should be understood that “administering” the composition can be understood to mean delivering or causing to be delivered the composition into the body of a patient. For example, “administering” can be understood to mean a healthcare professional prescribing, supervising, or managing the formal taking of the composition by a patient.

The composition of the invention may be provided in the form of two or more components, each containing the same composition. For example, the composition provided in the form of two sachets, each contain as a half quantity of the total amount of the composition. The amount of the composition in each sachet may be adjusted or tailored based on the individual patient's need.

In one embodiment, the dosage regimen for cleansing the gastrointestinal tract prior to endoscopy, colon surgery or gastrointestinal tract surgery using a single sachet-containing total dose of the composition comprises the steps of dissolving the total content of the sachet in water followed by drinking said mixture, either immediately or at intervals.

In a further embodiment, the dosage regimen for cleansing the gastrointestinal tract prior to endoscopy, colon surgery or gastrointestinal tract surgery includes using two sachets. Each sachet contains a part dose of the composition. The dosage regimen comprises the steps of: (a) dissolving the contents of the first sachet in water followed by drinking the mixture, and (b) dissolving the contents of the first sachet in water after a time period followed by drinking the mixture.

The methods of the invention may also include administering additional agents to the patient. For example, for added potency in certain clinical applications, a bowel stimulant such as biscodyl, or other agent known for its laxative properties may be taken in conjunction with the administration of these compositions as appropriate.

The composition may contain at least one pharmaceutical carrier according to conventional pharmaceutical techniques. The carrier may take a wide variety of forms depending on the form of preparation desired for administration. For example, for liquid oral preparations such as suspensions, elixirs and solutions, suitable carriers and additives include water, glycols, oils, alcohols, flavoring agents, preservatives, coloring agents and the like. For solid oral preparations such as powders, capsules and tablets, suitable carriers and additives include starches, sugars, diluents, granulating agents, lubricants, binders, disintegrating agents and the like. In some embodiments, the composition may include optional additives such as antioxidants, amino acids, caffeine, emulsifiers, minerals, micronutrients, phytochemicals (“phytonutrients”), stabilizers, thickening agents, medicaments, vitamins, or mixtures thereof.

### EXAMPLE 1

### Formulation for Mixing with 1 to 1.5 Liters of an Aqueous Solution

| Excipient | Amount (grams) |
|---|---|
| Polyethylene Glycol | 25-350 |
| Sodium Picosulfate | 40-200 |
| Electrolyte(s) | As needed |
| Optional Inorganic or | As needed |
| Organic Acid(s) | |
| Optional Carrier | As needed |
| Optional Additives | As needed |

Embodiments of the invention are also directed to a method of cleansing the gastrointestinal tract prior to an endoscopy including a colonoscopy or sigmoidoscopy, a barium enema examination, a capsule endoscopy, colon surgery or gastrointestinal tract surgery. The method comprises orally administering to the patient the pharmaceutical composition or aqueous solution as substantially described herein.

What is claimed is:

1. A gastrointestinal tract cleansing pharmaceutical composition for cleansing at least a portion of the gastrointestinal tract and for mixing with up to 1.5 liters of an aqueous solution, the composition comprising an admixture of: (a) polyethylene glycol, (b) sodium picosulfate; and (c) one or more electrolytes.

2. The gastrointestinal tract cleansing pharmaceutical composition of claim 1, wherein the composition consists essentially of an admixture of: (a) polyethylene glycol, (b) sodium picosulfate; and (c) one or more electrolytes.

3. The gastrointestinal tract cleansing pharmaceutical composition of claim 1, wherein the composition consists of an admixture of: (a) polyethylene glycol, (b) sodium picosulfate; and (c) one or more electrolytes.

4. The pharmaceutical composition of claim 1, wherein the composition is provided in a single packed dose.

5. The pharmaceutical composition of claim 1, wherein the composition is provided in the form of one or more of a powder, granules, pills, sachets, capsules, tablets, gel-caps, gel-caps filled with a paste or suspension.

6. The pharmaceutical composition of claim 1, wherein the one or more electrolytes are selected from the group consisting of sodium chloride, potassium chloride, sodium hydrogen carbonate, sodium sulfate, sodium carbonate, salts of magnesium, such as magnesium sulfate or magnesium citrate.

7. The pharmaceutical composition of claim 1, wherein the amount of polyethylene glycol in the composition is present in an amount of about 25 gm to about 350 gm.

8. The pharmaceutical composition of claim 1, wherein the amount of sodium picosulfate in the composition is present in an amount of about 40 gm to about 200 gm.

9. The pharmaceutical composition of claim 1, wherein the inorganic acid and/or organic acid are selected from the group consisting of citric acid, acetic acid, ascorbic acid, phosphoric acid, malic acid, succinic acid, formic acid, fumaric acid, maleic acid, adipic acid, butyric acid, glyconic acid, lactic acid, oxalic acid, tartaric acid and mixtures thereof.

10. The pharmaceutical composition of claim 1, wherein the composition is devoid of ascorbic acid, one or more salts of ascorbic acid, and mixtures thereof.

11. The pharmaceutical composition of claim 1, wherein the composition further comprises an alkali metal or alkaline earth metal sulphate or a mixture of alkali metal or alkaline earth metal sulphates.

12. A method of cleansing the gastrointestinal tract prior to an endoscopy procedure selected from one or more of colonoscopy, sigmoidoscopy, a barium enema examination, capsule endoscopy, colon surgery or gastrointestinal tract surgery, wherein the method comprises orally administering the pharmaceutical composition of claim 1.

13. An aqueous solution having a volume of less than 1.5 liters prepared by reconstituting, mixing and/or dissolving the pharmaceutical composition of claim 1 in an aqueous solvent or water.

14. An aqueous solution of up to 1.5 liter volume for cleansing the gastrointestinal tract, the aqueous solution comprising: (a) polyethylene glycol; (b) sodium picosulfate; and (c) one or more electrolytes.

15. The aqueous solution of claim 14, wherein the solution has a pH in the range of from about 3.0 to about 6.0.

16. The aqueous solution of claim 14, wherein the solution has a pH in the range of about 3.0 to about 4.0.

17. The aqueous solution of claim 14, wherein the solution has total volume of about 1 liter.

18. The aqueous solution of claim 14, wherein the solution has an osmolarity within the range of from 300 to 700 mOsmol/kg.

19. A method of cleansing the gastrointestinal tract prior to an endoscopy procedure selected from colonoscopy, sigmoidoscopy, a barium enema examination, capsule endoscopy, colon surgery or gastrointestinal tract surgery, wherein the method comprises orally administering the aqueous solution of claim 14 in a single dose or multiple doses.
